(19) United States
(12) Patent Application Publication
(10) Pub. No.: US 2015/0317908 A1
(43) Pub. Date: Nov. 5, 2015

(54) PATIENT REPORTED OUTCOME INSTRUMENTS FOR ENDOMETRIOSIS

(71) Applicant: BAYER PHARMA AKTIENGESELLSCHAFT, Berlin (DE)

(72) Inventors: Kamonthip Wichmann, Berlin (DE); Christoph Gerlinger, Berlin (DE); Christian Seitz, Zeuthen (DE); Adam Gater, Cheshire (GB)

(21) Appl. No.: 14/700,999

(22) Filed: Apr. 30, 2015

Related U.S. Application Data
(60) Provisional application No. 61/986,425, filed on Apr. 30, 2014.

(51) Int. Cl. G09B 7/00; A61K 31/192

(52) U.S. Cl. CPC G09B 7/00; A61K 31/192

(57) ABSTRACT

The present invention relates to methods of diagnosing, treating, and monitoring endometriosis utilizing patient reported outcome instruments.

### BACKGROUND OF THE INVENTION

1. Field of the Invention

The present invention relates to methods of diagnosing, treating, and monitoring endometriosis using printed and/or electronic patient reported outcome instruments.

2. Description of the Art

Endometriosis is a benign, estrogen-dependent gynecological disorder affecting 6 to 10% of women in their reproductive years. The disease is characterized by the presence of endometrium-like tissue outside the uterine cavity (endometriotic lesions). Dysmenorrhea (heavy menstrual bleeding), chronic pelvic pain, painful intercourse, and infertility are the most common symptoms of endometriosis, which can have a significant effect on patients' lives such as substantial morbidity, multiple operations, and decreased quality of life.

The gold standard in establishing the diagnosis of pelvic endometriosis is surgical assessment by laparoscopy or laparotomy. These circumstances and the variety of clinical manifestations typically lead to unspecific treatment and a diagnostic delay of 8 to 11 years of endometriosis.

Rather than by surgical assessment, endometriosis may be diagnosed and monitored by assessing the change in the key symptoms of endometriosis (e.g., pain).

However, no direct correlation exists between stage of disease, type of endometriotic lesion, and frequency and severity of symptoms, such as pain. Thus, in the absence of direct biomarkers, and reflecting the inherently subjective nature of the symptoms, endometriosis may be diagnosed and monitored by the patients themselves.

How to measure endometriosis-associated pelvic pain was addressed in Gerlinger et al. (Journal of Endometriosis 2012; 4(3): 109-116) comparing patient's response on the different scales Visual Analog Scale, Biberoglu and Behrman Score and SF-36 Bodily Pain Subscale with Clinical Global Impression score. A general measure of endometriosis-related pain can be recommended as primary endpoint in clinical trials to assess painful symptoms of endometriosis. In addition, a disease-specific quality of life tool is recommended to help interpret impact on patients' daily activities.

Patient-Reported Outcomes (PROs) are reports of the status of a patient's health condition that come directly from the patient, without interpretation of the patient's responses by a clinician or anyone else. Use of PROs is advised when measuring a symptom, sign, or state of disease best known by the patient or best measured from the patient perspective. PROs can be measured in absolute terms (e.g., severity of a symptom, sign, or state of a disease) or as a change from a previous measures.

PRO instruments for pain include pain diaries and pain scales. Older diagnostic tools like monthly Biberoglu+ Behrman score (B+B) or monthly VAS are not accepted anymore because the recall period is too long. Moreover, a subjective endpoint like pain needs to be supported by secondary assessment of functional impact. Deal et al. (Health and Quality of Life Outcome 2010, 8:64) show that a 17-item Endometriosis Pain and Bleeding Diary (EPBD) reliably and validly characterizes the types of pain identified by endometriosis patients. The EPBD was developed using a qualitative process that included clinician input, focus groups, and cognitive interviews.

### BRIEF SUMMARY OF THE INVENTION

In a first aspect, it is an object of the present invention to provide a patient reported outcome questionnaire (PRO) for patients who have or are suspected of having endometriosis.

In a second aspect, it is an object of the present invention to provide a PRO as essentially as shown in FIG. 1.

In a third aspect, it is an object of the present invention to provide a PRO as essentially as shown in FIG. 2.

In a fourth aspect, it is an object of the present invention to provide a PRO that measures endometriosis-related pain and comprises a diagram the clearly highlights the areas in which patients with endometriosis typically experience pain.

In a fifth aspect, it is an object of the present invention to provide a PRO that measures both chronic and acute endometriosis-related pain on a multi-point scale.

In a sixth aspect, it is an object of the present invention to provide a method of diagnosing a patient suffering from endometriosis comprising the steps of: providing a patient with a PRO who have or are suspected of having endometriosis.

In a seventh aspect, it is an object of the present invention to provide a method of treating a patient suffering from endometriosis comprising the steps of: providing a patient with a PRO who have or are suspected of having endometriosis, and administering a treatment for endometriosis.

In an eighth aspect, it is an object of the present invention to provide a method of monitoring the treatment of a patient suffering from endometriosis comprising the steps of: providing a patient with a first PRO adapted for patients who have or are suspected of having endometriosis, administering a treatment for endometriosis, providing the patient with a second PRO who have or are suspected of having endometriosis.

In a ninth aspect, it is an object of the present invention to provide a method of diagnosing, treating, or monitoring the treatment of endometriosis with a PRO adapted for patients who have or are suspected of having endometriosis, wherein the PRO instrument measures endometriosis-related pain and comprises a diagram the clearly highlights the areas in which patients with endometriosis typically experience pain.

In a tenth aspect, it is an object of the present invention to provide a method of diagnosing, treating, or monitoring the treatment of endometriosis with a PRO adapted for patients who have or are suspected of having endometriosis, wherein the PRO instrument measures both chronic and acute endometriosis-related pain on a multi-point scale.

According, the present invention provides PRO instruments for diagnosing endometriosis, treating endometriosis, and monitoring endometriosis treatment. The PRO instruments are a daily pain assessment (the Endometriosis Symptom Diary, ESD), and a weekly functional impact scales (the Endometriosis Impact Scale, EIS).

In a eleventh aspect, it is an object of the present invention to provide a PRO wherein patient is reporting endometriosis symptoms occurring during the past 24 hours into an Endometriosis Symptom Diary (ESD) wherein the ESD is optionally in an electronic format. The ESD consists into 5 domains of investigation for assessing endometriosis-related pain. In other words, the ESD is a questionnaire divided in 5 domains of investigation wherein each domains is containing 1 to 3 questions. The domains of investigation are
Endometriosis pain, Constant pain, Short-term pain, Pain associated with menstruation, and Dyspareunia,
Pelvic pain severity, Dysmenorrhea, Analgesic use, Dyspareunia and Menstrual bleeding intensity,
Endometriosis pain, Vaginal bleeding, sexual intercourse pain, Pain medication.

The invention is directed to pain in the target area, namely Endometriosis pain located in the area shown in the diagram of FIG. 1, screen 2 [Instruction 3].

In another embodiment the domains of the ESD are:
Assessment of endometriosis pain at its worst,
Assessment of constant pain at its worst,
Assessment of short-term pain at its worst,
Assessment of menstrual bleeding intensity,
Assessment of pain during sexual intercourse,
Assessment of avoidance of sexual intercourse due to pain/anticipated pain,
Assessment of analgesic use.

In another embodiment the domains of the ESD consists into at least 5 domains of investigation for assessing endometriosis-related pain selected from the lists above.

In a twelfth aspect, it is an object of the present invention to provide PRO wherein patient is reporting impact of endometriosis symptoms onto life occurred the past seven days into an Endometriosis Impact Scale (EIS) wherein the EIS is optionally in an electronic format. The EIS consists into 6 domains of investigation for assessing impact of endometriosis-related pain onto patient life. In other words, the EIS is a questioner divided in 6 domains of investigation wherein each domains is containing 1 to 3 questions.

The domains of investigation are
Physical activities, Emotional well-being, Social and leisure activities, Work and study, Household activities and Sexual activities.

### BRIEF DESCRIPTION OF THE SEVERAL VIEWS OF THE DRAWING(S)

FIG. 1 is an Endometriosis Symptom Diary according to the present invention.

FIG. 2 is an Endometriosis Impact Scale according to the present invention.

FIG. 3 shows the domains of the ESD of FIG. 1.

FIG. 4 shows the domains of the EIS of FIG. 2.

FIG. 5 shows the observed average person measure for EIS Physical Activities Scale.

FIG. 6 shows the observed average person measure for EIS Emotional Well-being Scale.

### DETAILED DESCRIPTION OF THE INVENTION

The Endometriosis Symptom Diary (ESD) and Endometriosis Impact Scale (EIS) are self-administered questionnaires responded by the subjects themselves.

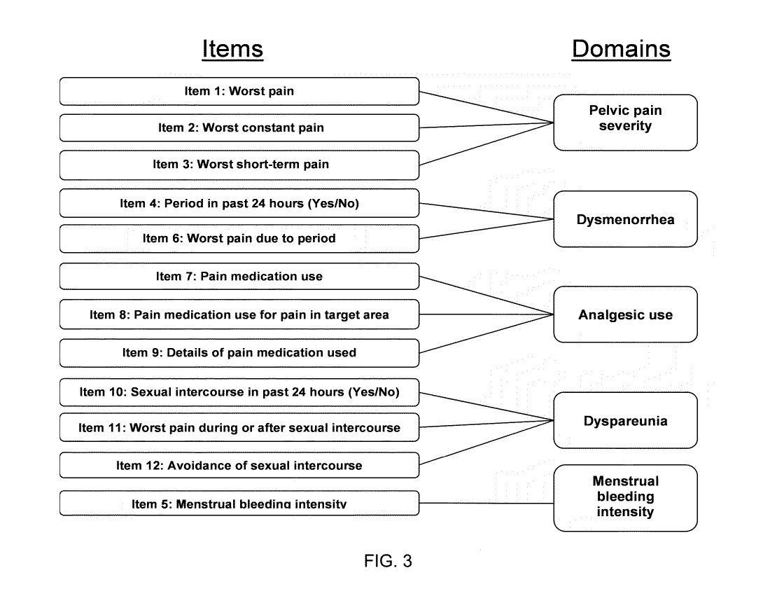

The ESD is a diary assessing patient experience of symptoms of endometriosis in several domains with a recall period of the past, e.g., 24 hours. Referring to FIG. 3, the domains of the ESD include pelvic pain severity, dysmenorrhea, analgesic use, dyspareunia, and menstrual bleeding severity. Items assess endometriosis-related pain using a 0-10 point numerical rating scale (NRS) The ESD may include documentation of concomitant intake of supportive pain medication. The ESD may be completed by respondents every evening (e.g., between 18:00 and 24:00).

The ESD preferably includes a diagram that clearly highlights the areas in which patients with endometriosis typically experience pain and which has been referred to as the target area. This definition maximizes the value of the diagram and also avoids the use of specific clinical terminology which is unlikely to be understood by patients.

The EIS is a questionnaire assessing impact of endometriosis symptoms over the pastseven days. Items assess impact on a 5-point Likert-type scale. The EIS is to be completed by respondents once every 7 days (e.g., between 18:00 and 24:00) starting at the screening visit 1 (Visit 1) and throughout the whole study period.

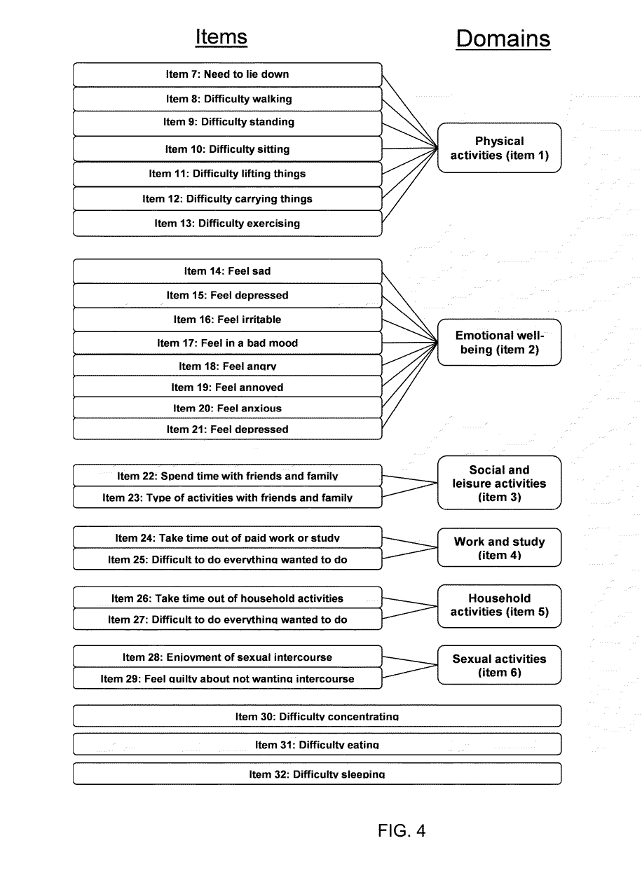

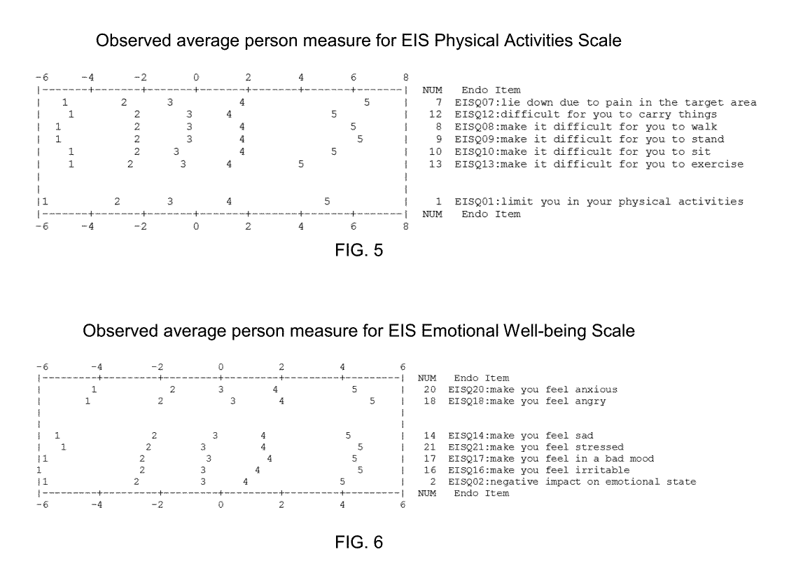

Referring to FIG. 4, the EIS may assess the impact of endometriosis in several domains or subscales, including Physical activities, Emotional well-being, Social and leisure activities, Work and study, Household activities, Sexual activities, Difficulty concentrating, Difficulty eating, and Difficulty sleeping. Physical activities assessed by the EIS include the need to lie down, difficulty walking, standing, or sitting, difficulty lifting or carrying things, and difficulty exercising. Emotions assessed by the EIS include feeling sad, depressed, irritable, angry, annoyed, anxious, or in a bad mood. Social and leisure activities assessed by the EIS include spending time with friends and family. Work and study activities assessed by the EIS include taking time off from paid work or study. Sexual activities assessed by the EIS include enjoyment of sexual activities and feeling guilty about avoiding sexual activities.

The development of the ESD and EIS was informed by qualitative and quantitative stages to establish content validity. The qualitative stage consisted of face-to-face concept elicitation and cognitive interviews. The quantitative stage utilized Rasch Model analyses to evaluate item performance based on baseline data obtained during a prospective, observational validation study. Cognitive interviewing of the ESD and EIS reveals that both measures can be well understood and consistently interpreted by participants. Rasch analyses revealed no disordering of the response categories for the items, and the full range is covered with no gaps in measurement of the respective concepts.

The ESD and EIS are preferably completed independently by the subject herself in a quiet, private area, accessible only to the subject and study staff, and before any other study-related procedures, including discussions with study staff, physical examinations, diary review, etc. The subject may be seated, comfortably, and allowed sufficient time to complete the questionnaires (i.e. time should be allocated at each visit to accommodate filling the questionnaires and the subject should not feel “rushed”). All questionnaires should be administered in the subject's local language. Study personnel preferably allow the subject privacy while completing the questionnaires but are available nearby in the event the subject has any technical questions. At no time should the site staff be allowed to respond other than for technical questions. To reduce the amount of missing data, site staffs are preferably instructed to check the questionnaire for completeness.

Any of the PRO instruments of the present invention may be provided in written (paper-based) or electronic form, including, without limitation, recordation on a computer readable medium, completion on a computer, including, in a web-based form or via telephonic interaction. In one embodiment, the PRO instruments are utilized in an interactive voice response (IVR) format. In one embodiment, the PRO instruments are utilized in a web-based or cloud-based format made available on a social media cite or otherwise. The answers can be recorded in similar formats, including in the form of paper-based or computer-readable reports.

Preferably, the ESD and EIS are collected using electronic devices, e.g. an electronic hand-held device at home and a tablet computer at the study site. The time point for data entry is technically regulated. Training on the use of the devices and ongoing technical support ensure minimization of failure in/missing of data entry. Sites staff are trained regarding the use of the electronic devices. Standardized technical training for the subjects is provided by the trained study site personnel. In addition, a 24-hour help desk is available. The subjects are asked to confirm their understanding on the use of the devices and completion of the questionnaires before the device is activated. The hand-held electronic devices are returned to the site as soon as a subject leaves the study. Data is uploaded to a data base in regular intervals. Completeness of hand-held and tablet entries is monitored by the investigator or dedicated staff via password-protected web access on a regular basis and at each visit.

Once one or both of the PRO instruments according to the present invention have been completed, a patient for treatment of endometriosis is identified based at least on pain perceived by the patient that impairs at least one function.

While there is no cure for endometriosis, there are two types of interventions; treatment of pain and treatment of endometriosis-associated infertility. In many women menopause (natural or surgical) will abate the process. In patients in the reproductive years, endometriosis is merely managed: the goal is to provide pain relief, to restrict progression of the process, and to restore or preserve fertility where needed. In younger women with unfulfilled reproductive potential, surgical treatment attempts to remove endometrial tissue and preserving the ovaries without damaging normal tissue.

Surgical treatment, typically performed via laparoscopy, consists of the excision of endometriosis lesions, adhesions, resection of endometriomas, and restoration of normal pelvic anatomy as much as is possible.

A hysterectomy (removal of the uterus) can be used to treat endometriosis in patients who do not wish to conceive, however this should only be done when combined with removal of the endometriosis by excision, as if endometriosis is not also removed at the time of hysterectomy, pain may still persist.

For patients with extreme pain, a presacral neurectomy may be very rarely performed where the nerves to the uterus are cut. However, this technique is almost never used due to the high incidence of associated complications including presacral haematoma and irreversible problems with urination and constipation.

The currently available pharmacological treatments of endometriosis are anti-inflammatory and hormonal. In the early stages of endometriosis non-steroidal anti-inflammatory drugs (NSAIDs), such as naproxen indomethacin, ibuprofen, mefenamic acid, and flurbiprofen, are often successful in relieving the pelvic pain.

Hormonal treatment is given mainly to inhibit the estrogen production by the ovaries. Various hormones are available for suppressing ovarian function including dienogest, progesterone and its derivatives, cyproterone acetate, desogestrel, etonogestrel, levonorgestrel, lynestrenol, medroxyprogesterone acetate, norethisterone, norethisterone acetate, norgestimate, drospirenone, gestodene, 19-nor-17-hydroxy progesterone esters, 17α-ethinyltestosterone and derivatives thereof, 17α-ethinyl-19-nor-testosterone and derivatives thereof, ethynodiol diacetate, dydrogesterone, norethynodrel, allylestrenol, medrogestone, norgestrienone, ethisterone and di-norgestrel; and androgenic steroids, such as danazol and gestrinone.

Other active substances that have been tested to treat endometriosis include inhibitors of leukotriene, e.g., zafirlukast and montelukast and its salts, oxytocin antagonists, pancreatic trypsin inhibitors like Trasylol, COX-inhibitors, antifibrinolytic drugs, such as tranexamic acid and precursors thereof, aminocapronic acid, PAI-I, desmopressin, clomiphene citrate, p-aminomethyl-benzoic acid, cytokine inhibitors, glucocorticoids, GnRH agonists; and aromatase inhibitors.

The present invention is also directed to methods of evaluating the effectiveness of a treatment regimen administered for endometriosis. The evaluation compares the patients most recent menstrual period with a previous period for which comparative data are available. The comparative data used for the evaluation include, for example: a) determining a patient's perceived endometriosis symptoms during their most recent menstrual period; b) determining how much the patient's perceived blood loss limited their work outside and inside the home; c) determining how much the patient's perceived blood loss limited their physical activities; d) determining how much the patient's perceived blood loss limited their social and leisure activities; e) determining the specific activities that were limited by the patient's blood loss; and f) determining the patient's perceived blood loss during the most recent menstrual period in comparison to the blood loss during the patient's previous menstrual period, g). determining the patient's change in measures of work limitation, measures of physical limitation, measures of social/leisure limitation, during the most recent menstrual period in comparison to the patient's previous menstrual period.

### Example 1

### Endometrial Symptom Diary (ESD)

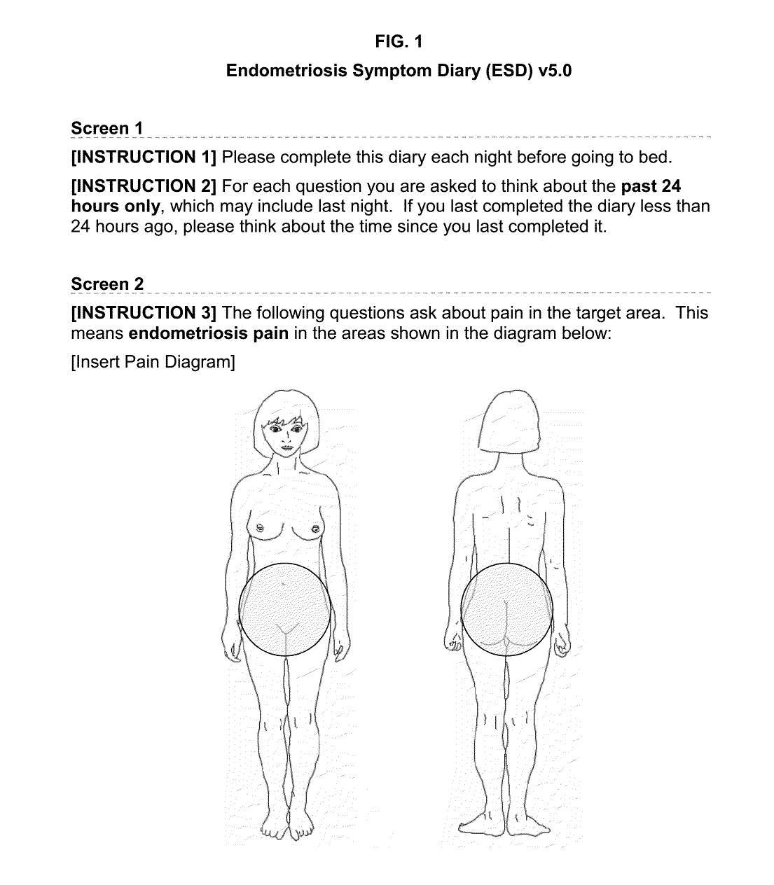

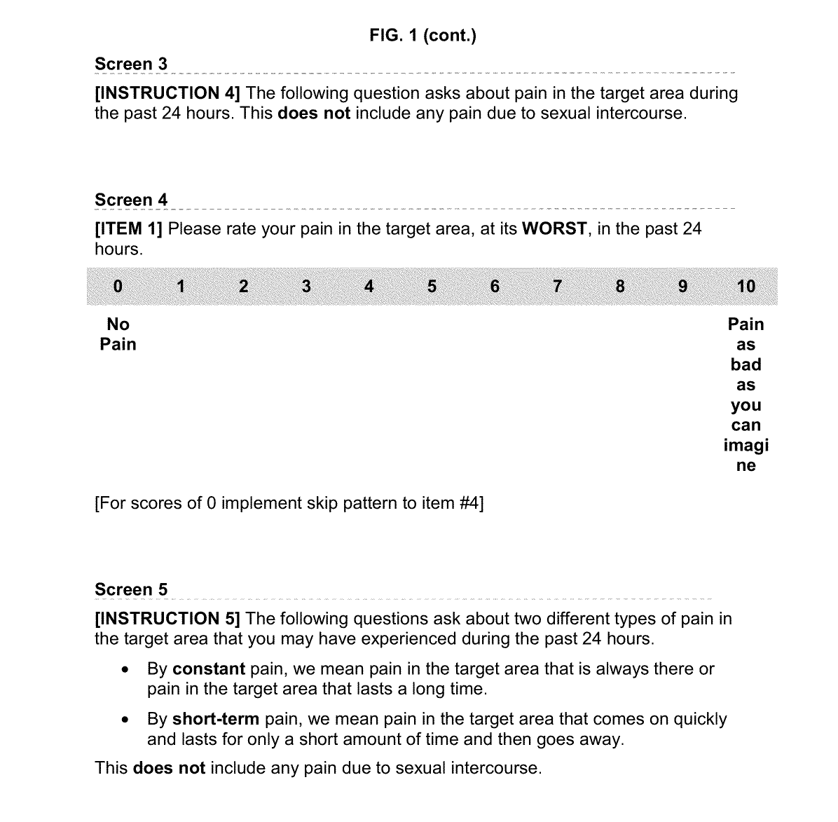

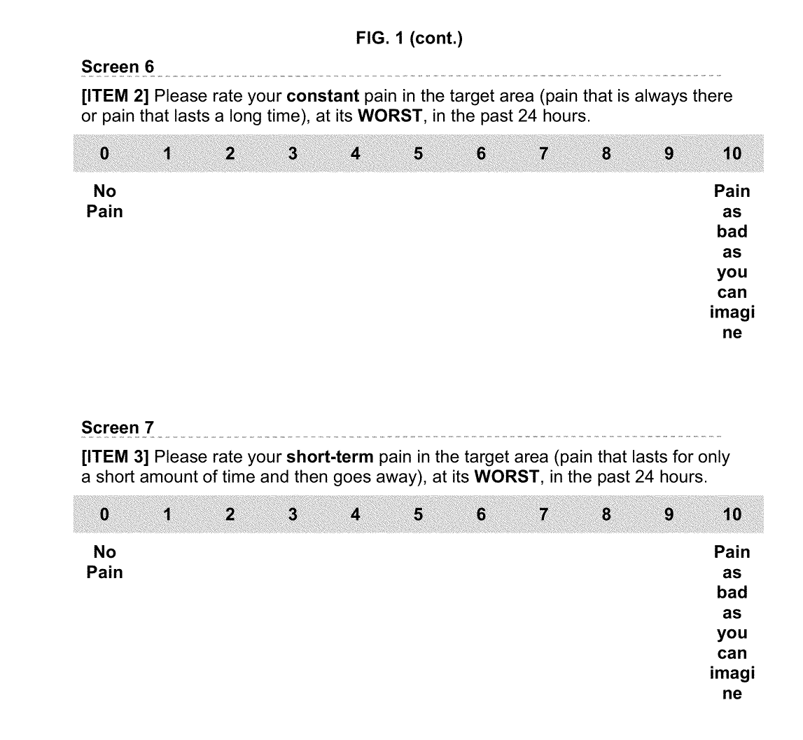

The Endometrial Symptom Diary of FIG. 1 is used as follows, in this Example:

On Screen 1 (Instructions 1 and 2), a subject is instructed to complete an ESD each night before going to bed. The subject is also instructed, for each question, to think about the past 24 hours only, which may include the previous night. If an ESD was last completed less than 24 hours ago, the subject is instructed to think about the time since the last ESD was completed.

On Screen 2 (Instruction 3), the subject is instructed to answer questions only about endometriosis pain in the target area, which means endometriosis pain in the areas shown in FIG. 1.

On Screen 3 (Instruction 4), the subject is instructed to answer questions only about pain in the target area within the last 24 hours (excluding pain due to sexual intercourse).

On Screen 4 (Item 1), the subject is asked to rate her pain in the target area, at its WORST, in the past 24 hours on a scale of 0 to 10, with 0 being no pain and 10 being pain as bad as the subject can imagine. For a score of 0, the ESD skips to Screen 8 (Item 4), which is a question about vaginal bleeding during the past 24 hours, as described below.

On Screen 5 (Instruction 5), the subject is instructed that subsequent questions ask about two different types of pain in the target area that she may have experienced during the past 24 hours. Constant pain means pain in the target area that is always there or pain in the target area that lasts a long time. Short-term pain means pain in the target area that comes on quickly and lasts for only a short amount of time and then goes away. Constant and short-term pains do not include any pain due to sexual intercourse.

On Screen 6 (Item 2), the subject is asked to rate her constant pain in the target area (pain that is always there or pain that lasts a long time), at its WORST, in the past 24 hours on a scale of 0 to 10, with 0 being no pain and 10 being pain as bad as the subject can imagine.

On Screen 7 (Item 3), the subject is asked to rate her short-term pain in the target area (pain that lasts for only a short amount of time and then goes away), at its WORST, in the past 24 hours on a scale of 0 to 10, with 0 being no pain and 10 being pain as bad as the subject can imagine.

On Screen 8 (Item 4), the subject is asked to select the response that best describes her vaginal bleeding during the past 24 hours. The answer choices are: None (no vaginal bleeding), Spotting (less than associated with normal menstruation with no need for sanitary protection, except for panty liners), Light (less than associated with normal menstruation with need for sanitary protection), Normal (like normal menstruation), and Heavy (more than normal menstruation)

Screen 9 (Item 5) asks the subject whether the subject had sexual intercourse in the past 24 hours. If the answer to Item 5 is “yes,” the ESD proceeds to Screen 10 (Item 6), which is a follow up question about pain during sexual intercourse, but skips Screen 11 (Item 7), which is a question about avoiding sexual intercourse. If the answer to Item 5 is “no,” the ESD skips Item 6 and proceeds to Item 7.

Only if the subject answers “yes” to Item 5 on Screen 9 regarding sexual intercourse in the past 24 hours, Screen 10 (Item 6) asks the subject to rate her WORST pain in the target area either during or after sexual intercourse on a scale of 0 to 10, with 0 being no pain and 10 being pain as bad as the subject can imagine.

Only if the subject answers “no” to Item 5 on Screen 9 regarding sexual intercourse in the past 24 hours, Screen 11 (Item 7) asks the subject whether, during the past 24 hours, she avoid sexual intercourse because of pain or expected pain in the target area. The subject must answer either “yes” or “no.”

Screen 12 (Instruction 6) instructs the subject that the following questions ask about any pain medications she took in addition to the study medication (if any) in the past 24 hours.

On Screen 13 (Item 8), the subject is asked whether she took any pain medication, other than the study medication in the past 24 hours. The subject must answer either “yes” or “no.” If the answer to Item 8 is “yes,” the ESD proceeds to Screens 14 (Item 9) and 15 (Items 10a and 10b), which are follow up questions about the pain medication taken by the subject. If the answer to Item 8 is “no,” the ESD finishes. Screen 14 (ITEM 9) asks the subject whether she took the other pain medication to reduce pain in the target area. The subject must answer either “yes” or “no.”

Finally, on Screen 15 (Items 10a and 10b) the subject is asked how many supportive pain medications distributed in trial, e.g., ibuprofen, did she take in the past 24 hours. The patient must enter the number of tablets and the tablets unit strength (e.g., 400 mg). Screen 15 also asks whether the subject took any other pain medication in the past 24 hours. The subject must answer either “yes” or “no.”

### Example 2

### Endometrial Impact Scale (EIS)

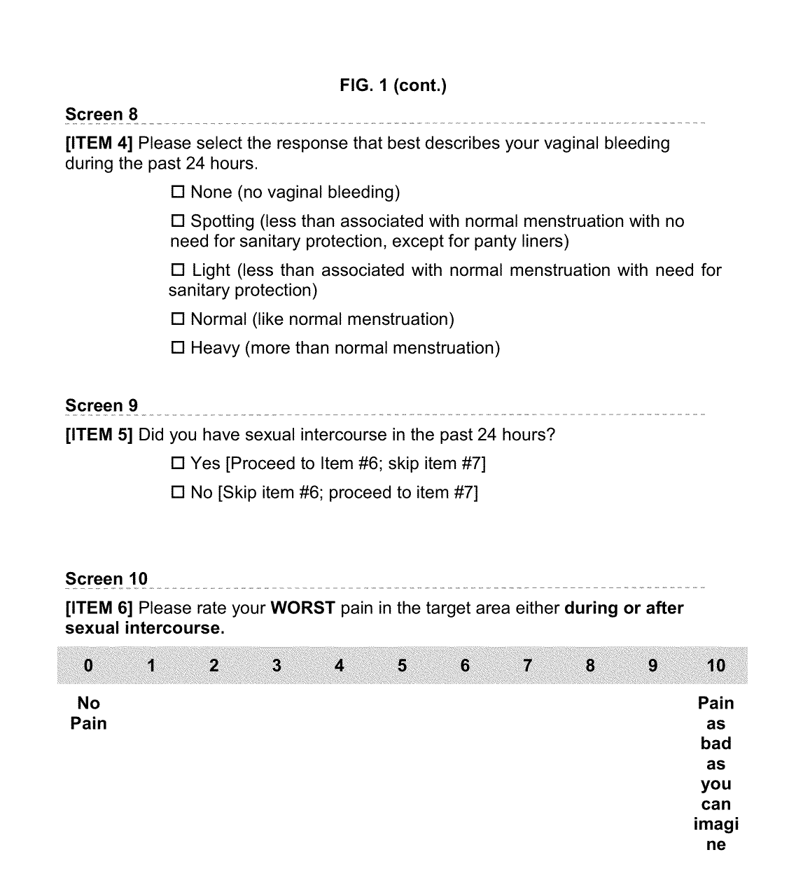

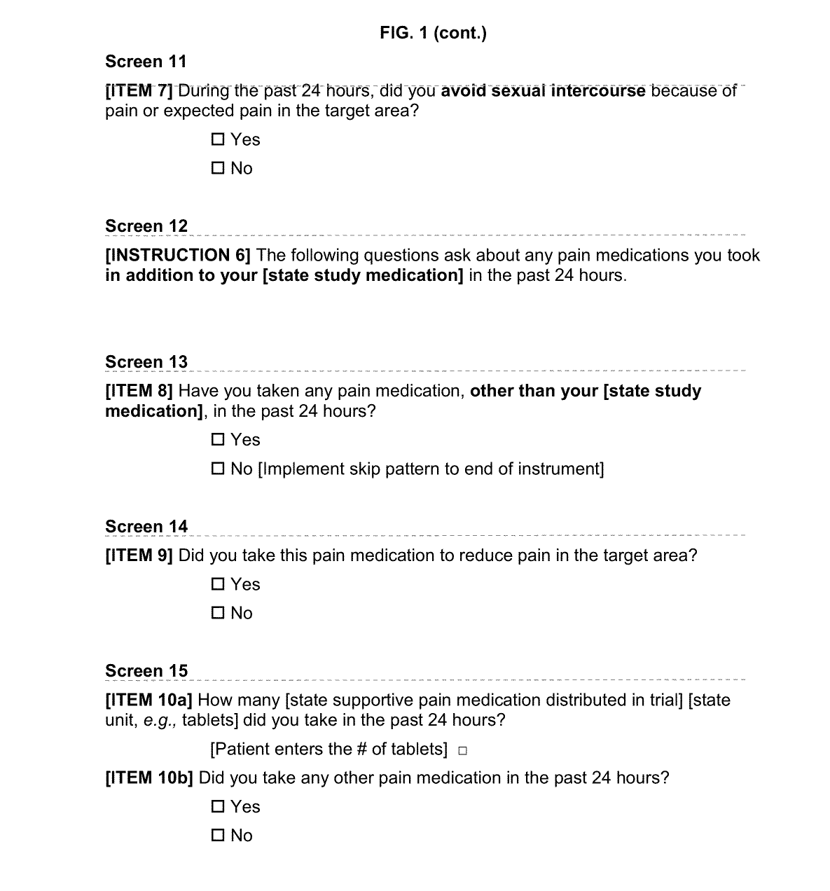

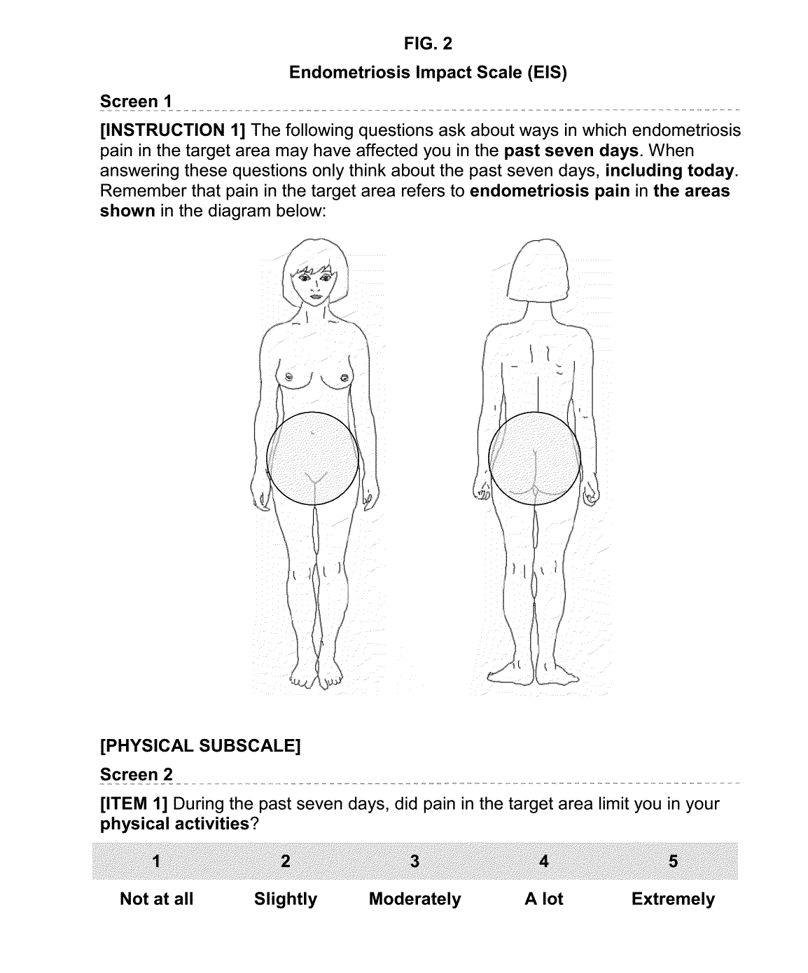

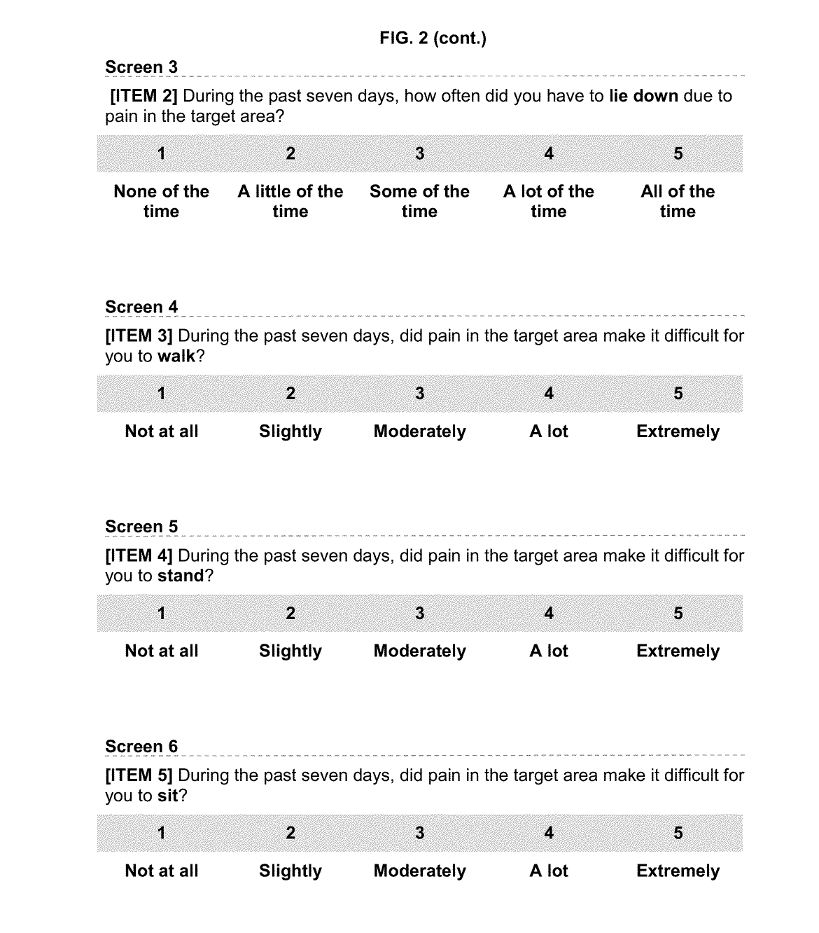

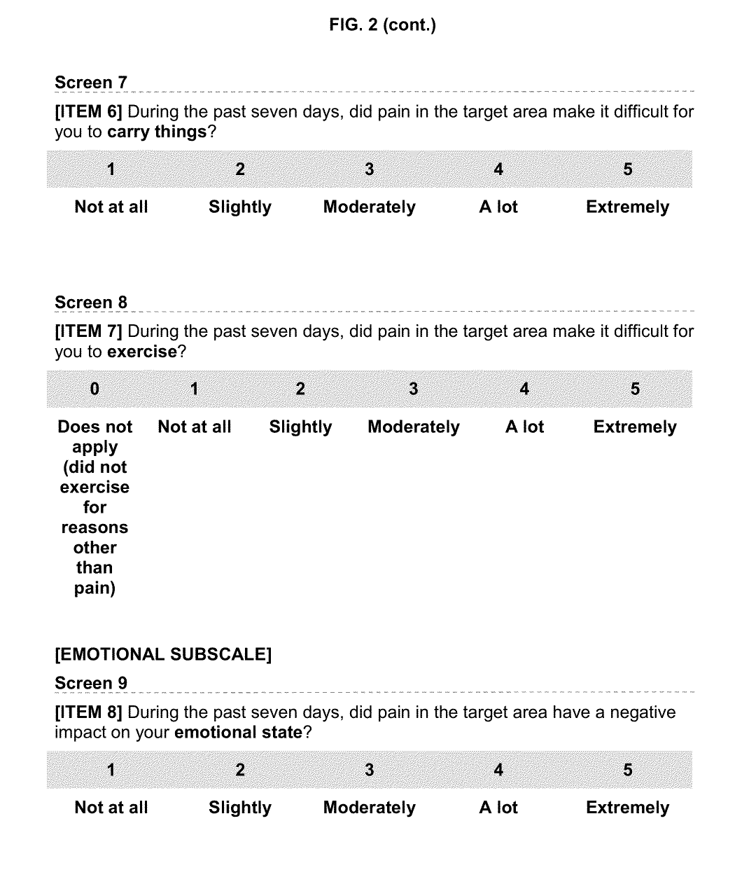

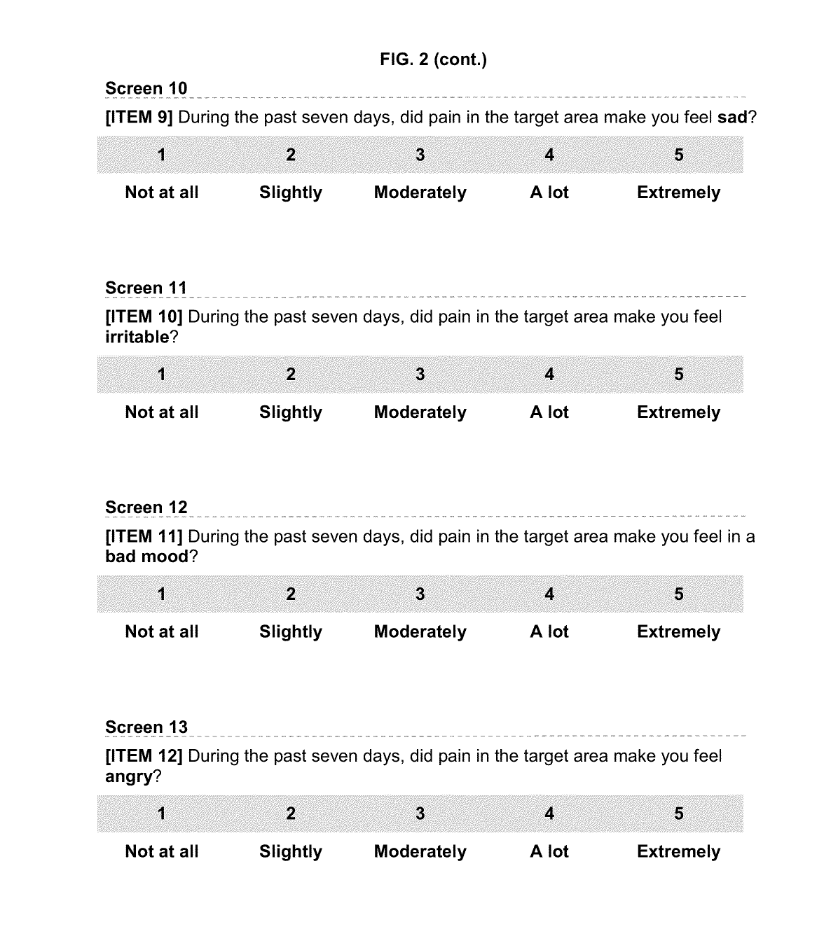

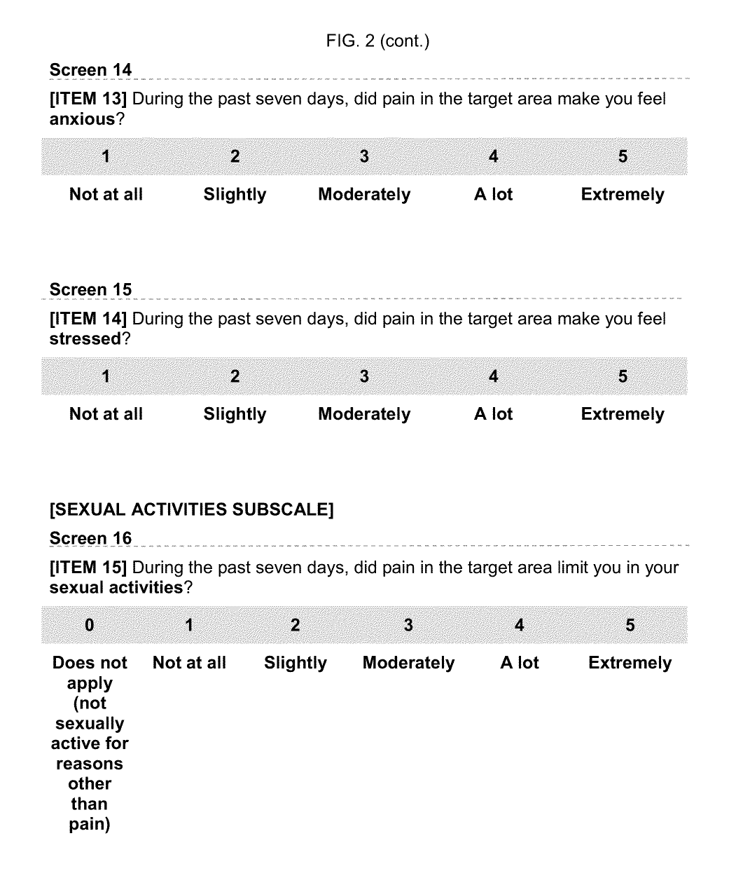

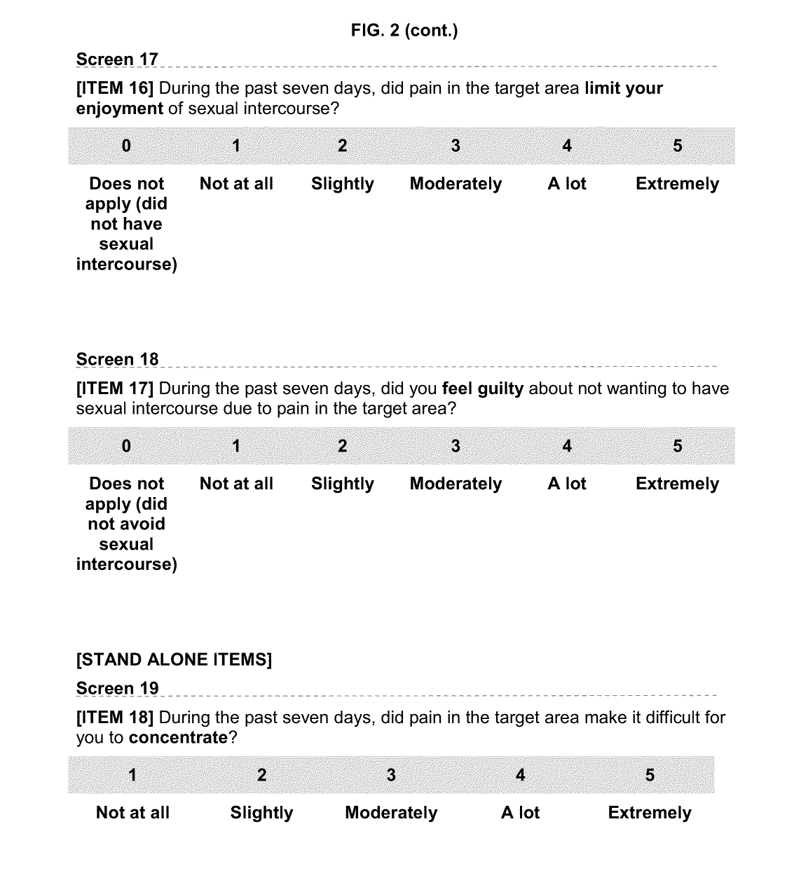

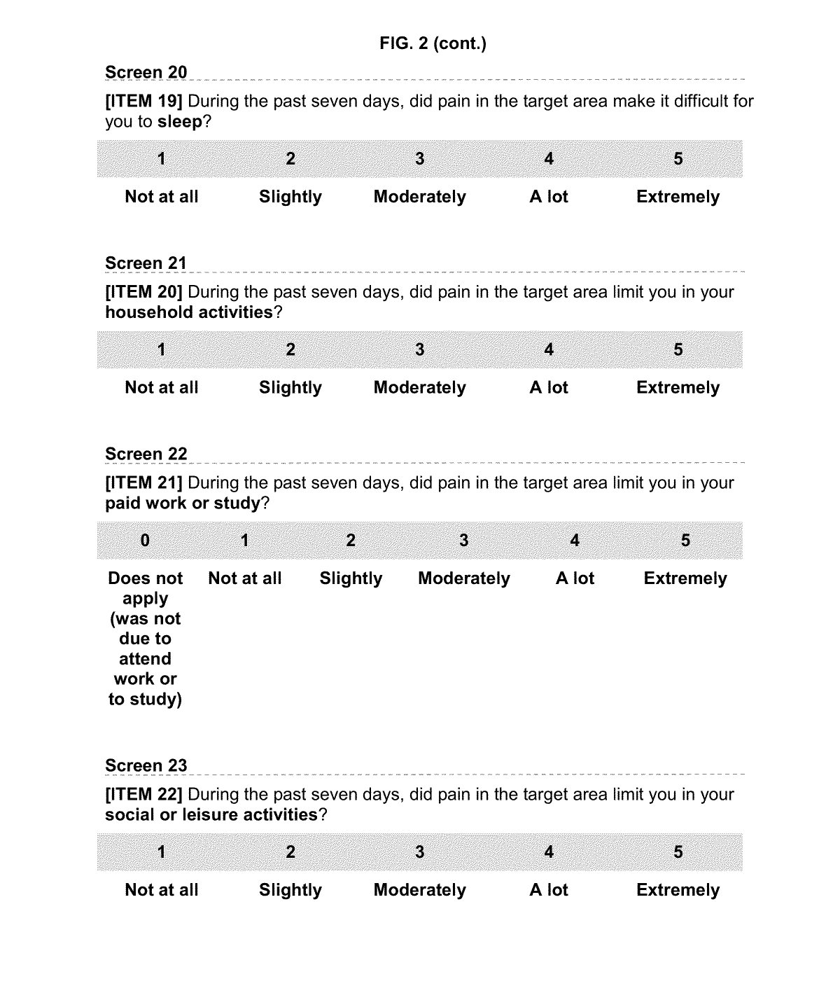

The Endometrial Impact Scale of FIG. 2 is used as follows, in this Example:

Screen 1 (Instruction 1) instructs the subject that the following questions ask about ways in which endometriosis pain in the target area may have affected her in the past seven days. When answering these questions, the subject is instructed to only think about the past seven days, including the day of the EIS, and to remember that pain in the target area refers to endometriosis pain in the areas shown in the diagram.

The EIS is divided into three sub-scales. The first subscale is the Physical Subscale. The second subscale is the Emotional Subscale. The third subscale is the Sexual Subscale. The EIS may include stand-alone items not covered in the subscales.

The Physical Subscale may comprises seven items (i.e., questions) shown on seven screens according to the following Table 1.

TABLE 1
Physical Subscale

| Screen | Item | Questions |
|---|---|---|
| 2 | 1 | During the past seven days, did pain in the target |
| | | area limit you in your physical activities? |
| 3 | 2 | During the past seven days, how often did you have |
| | | to lie down due to pain in the target area? |
| 4 | 3 | During the past seven days, did pain in the target |
| | | area make it difficult for you to walk? |
| 5 | 4 | During the past seven days, did pain in the target |
| | | area make it difficult for you to stand? |
| 6 | 5 | During the past seven days, did pain in the target |
| | | area make it difficult for you to sit? |
| 7 | 6 | During the past seven days, did pain in the target |
| | | area make it difficult for you to carry things? |
| 8 | 7 | During the past seven days, did pain in the target |
| | | area make it difficult for you to exercise? |

Items 1 and 3-7 of the Physical Subscale must be answered by the subject on a scale of 1 to 5 with 1 being “Not at all” and 5 being “Extremely” according to the following Table 2.

TABLE 2
Scoring of Items 1 and 3-7 of the Physical Subscale

| 1 | 2 | 3 | 4 | 5 |
|---|---|---|---|---|
| Not at all | Slightly | Moderately | A lot | Extremely |

Item 2 of the Physical Subscale must be answered on a scale of 1 to 5 with 1 being “None of the Time” and 5 being “All of the Time” according to the following Table 3.

TABLE 3
Scoring of Item 2 of the Physical Subscale

| 1 | 2 | 3 | 4 | 5 |
|---|---|---|---|---|
| None of the | A little of the | Some of the | A lot of the | All of the |
| time | time | time | time | time |

The Emotional Subscale may comprises seven items (i.e., questions) shown on seven screens according to the following Table 4.

TABLE 4
Emotional Subscale

| Screen | Item | Questions |
|---|---|---|
| 9 | 8 | During the past seven days, did pain in the target area |
| | | have a negative impact on your emotional state? |
| 10 | 9 | During the past seven days, did pain in the target area |
| | | make you feel sad? |
| 11 | 10 | During the past seven days, did pain in the target area |
| | | make you feel irritable? |
| 12 | 11 | During the past seven days, did pain in the target area |
| | | make you feel bad mood? |
| 13 | 12 | During the past seven days, did pain in the target area |
| | | make you feel angry? |
| 14 | 13 | During the past seven days, did pain in the target area |
| | | make you feel anxious? |
| 15 | 14 | During the past seven days, did pain in the target area |
| | | make you feel stressed? |

Items 8-14 of the Emotional Subscale must be answered by the subject on a scale of 1 to 5 with 1 being “Not at all” and 5 being “Extremely” according to the following Table 5.

TABLE 5
Scoring of Item 8-14 of the Emotional Subscale

| 1 | 2 | 3 | 4 | 5 |
|---|---|---|---|---|
| Not at all | Slightly | Moderately | A lot | Extremely |

The Sexual Subscale may comprise three items (i.e., questions) shown on three screens according to the following Table 6.

TABLE 6
Sexual Subscale

| Screen | Item | Questions |
|---|---|---|
| 16 | 15 | During the past seven days, did pain in the target area |
| | | limit you in your sexual activities? |
| 17 | 16 | During the past seven days, did pain in the target area |
| | | limit your enjoyment of sexual intercourse? |
| 18 | 17 | During the past seven days, did you feel guilty about |
| | | not wanting to have sexual intercourse due to pain in |
| | | the target area? |

Items 15-17 of the Sexual Subscale must be answered by the subject on a scale of 1 to 5 with 1 being “Not at all” and 5 being “Extremely” according to the following Table 7.

TABLE 7
Scoring of Items 15-17 of the Sexual Subscale

| 1 | 2 | 3 | 4 | 5 |
|---|---|---|---|---|
| Not at all | Slightly | Moderately | A lot | Extremely |

In addition to the Physical, Emotional, and Sexual Subscales, the EIS may comprise stand-alone items that do not fit within one of the three subscales.

For example, the Stand-Alone Items may comprise five items (i.e., questions) shown on five screens according to the following Table 8.

TABLE 8
Stand-Alone Items

| Screen | Item | Questions |
|---|---|---|
| 19 | 18 | During the past seven days, did pain in the target |
| | | area make it difficult for you to concentrate? |
| 20 | 19 | During the past seven days, did pain in the target |
| | | area make it difficult for you to sleep? |
| 21 | 20 | During the past seven days, did pain in the target |
| | | area limit you in your household activities? |
| 22 | 21 | During the past seven days, did pain in the target |
| | | area limit you in your paid work or study? |
| 23 | 22 | During the past seven days, did pain in the target |
| | | area limit you in your social or leisure activities? |

Items 18-22 of the Stand-Alone Items must be answered by the subject on a scale of 1 to 5 with 1 being “Not at all” and 5 being “Extremely” according to the following Table 9.

TABLE 9
Scoring of Stand-Alone Item 18-22

| 1 | 2 | 3 | 4 | 5 |
|---|---|---|---|---|
| Not at all | Slightly | Moderately | A lot | Extremely |

### Example 3

### Recall Period for the EIS

Use of the most appropriate recall period is critical for the content validity of PROs instruments. The most appropriate recall period depends on the PRO's concept of interest and intended context of use and requires balancing maximization of data quality while minimizing respondent burden. For a PRO instrument that assesses the impact of endometriosis pain, the appropriateness of a “7-day” recall period (versus a “24-hour” recall period) may be investigated as follows.

Data was collected during an observational study for the quantitative validation for the newly developed Endometriosis Impact Scale (EIS). Participants completed 8 items regarding the impact of endometriosis pain on their ability to perform physical activities (5-point scale). Items were administered daily for 28 days with a 24-hour recall and also at the end of each 7-day period with 7-day recall. Pearson correlations were calculated for each item between the item scores of the 7-day recall and the average of the daily item scores over the same periods. Items scores were also correlated with those from the previous day and with the worst score during the 7-day period, to assess potential “recency” and “saliency” effects respectively. Differences between the item scores of the 7-day recall and the average daily item scores over time were also examined.

Correlations between item scores of the 7-day recall and mean daily scores over the same period were excellent for all 8 items (r=0.84-0.91). Further, these correlations were higher than the correlations with the item scores from the previous day (r=0.73-0.81) or for the worst day during the same period (r=0.60-0.76). The 7-day recall item scores were slightly higher than the daily average scores, but equivalent in detecting change over time.

### Example 5

### Item Evaluation and Determination of Scoring Algorithms (VALEPRO)

As shown in the following Table 10, diverse samples of women with endometriosis, representative of the population to be included in planned clinical trials, were recruited for patient research.

TABLE 10
Demographic and clinical characteristics of study samples
used to establish the content validity of the ESD and EIS

| | Concept | Cognitive | Validation |
|---|---|---|---|
| Demographic and clinical | Elicitation | Debriefing | Study |
| characteristics | (n = 45) | (n = 31) | (n = 268) |
| Age | | | |
| Mean (Range) | 33.9 (18-44) | 36.3 (21-45) | 31.6 (19-45) |
| Ethnic Background n (%) | | | |
| Caucasian or White | 10 (66.7) | 8 (42.1) | 241 (89.9) |
| African American | 3 (20.0) | 9 (47.4) | 22 (8.2) |
| Other | 2 (13.3) | 2 (10.5) | 5 (1.9) |
| Education Level n (%) | | | |
| Elementary education | 0 (0.0) | 0 (0.0) | 16 (6.0) |
| High school diploma | 4 (8.9) | 3 (9.7) | 111 (41.4) |
| or GED | | | |
| Some years of college | 8 (17.8) | 5 (16.1) | — |
| Certificate program | 7 (15.6) | 9 (29.0) | — |
| College or University | 1 (2.2) | 10 (32.3) | 113 (42.2) |
| degree | | | |
| Graduate or professional | 25 (55.6) | 4 (12.9) | — |
| degree | | | |
| Experience of pain at | 0 (0.0) | 1 (3.2)^{†} | 1 (0.4) |
| screening n (%)* | | | |
| No pain (0) | 16 (35.6) | 9 (29.0)^{†} | 67 (25.0) |
| Mild (1-4) | 14 (31.1) | 8 (25.8) | 77 (28.8) |
| Moderate (5-6) | 15 (33.3) | 13 (41.9) | 107 (39.9) |
| Severe (7-10) | 5.8 (3-10) | 5.9 (0-10) | 5.9 (0-10) |
| Mean (range) | | | |
| Current treatment n (%) | | | |
| Painkillers | 22 (48.9) | 19 (61.3) | 109 (40.7) |
| Oral contraceptives | 13 (28.9) | 11 (35.5) | 48 (17.9) |
| Other hormonal contra- | 3 (6.7) | 5 (16.1) | 7 (2.6) |
| ceptives | | | |
| GnRH analogue | 4 (8.9) | 5 (16.1) | 13 (4.9) |
| Progestin | 3 (6.7) | 1 (3.2) | 40 (14.9) |
| Other | 3 (6.7) | 1 (3.2) | 23 (8.6) |

Data not available for European sample; therefore percentages calculated based on US sample only (concept elicitation: n=15, cognitive debriefing: n=19)

*Self-rated assessment of endometriosis-associated pain at its worst in the last 24 hours (using 0-10 NRS where 0=no pain and 10=pain as bad as you can imagine) Counts not mutually exclusive

Referring to FIGS. 3 and 4, item descriptive statistics demonstrated that all ESD and EIS response options were utilized by participants with no significant floor or ceiling effects and the percentage of missing data was low, supporting the validity of the respective instrument response options.

Inter-item correlations of the three main endometriosis pain items in ESD ranged from 0.80 to 0.91. Inter-item correlation in the EIS revealed that the majority of correlations were low-moderate (r<0.70), and there was evidence of clusters with moderate-high (r=0.70-0.79) and very high (r=0.80) correlations

Based on eigenvalues (three eigenvalues were above 1: 19.79, 2.07, and 1.32) and the screen plot from the EFA, three domains for the EIS were identified: ‘Physical activities’, ‘Emotional well-being’ and ‘Sexual activities’. Factor loadings are showed in Table 11.

No gaps were observed in Rasch item maps for the ESD and EIS suggesting that items adequately cover the full continuum of endometriosis symptom and impact severity.

Observation of Rasch-Andrich thresholds (step parameters) supported the validity of ESD and EIS response options with no evidence of response disordering. FIGS. 3 and 4 show the observed average measures (i.e. the average impact severity of participants who chose each response option) for items in EIS Physical Activities domain and Emotional Well-Being domain.

TABLE 11
Exploratory Factor Analysis: Standardized Regression Coefficients

| | Factor 1 | Factor 2 | Factor 3 |
|---|---|---|---|
| | (Physical | (Emotional Well- | (Sexual |
| Item | Activities) | being) | Activities) |
| 1. Physical activities | 0.76 | 0.07 | 0.15 |
| (global) | | | |
| 7. Lie down | 0.67 | 0.19 | 0.05 |
| 8. Walk | 0.78 | 0.21 | −0.04 |
| 9. Stand | 0.78 | 0.23 | −0.06 |
| 10. Sit | 0.71 | 0.23 | −0.18 |
| 11. Lift | 0.85 | 0.06 | 0.00 |
| 12. Carry | 0.79 | 0.09 | 0.02 |
| 13. Exercise | 0.81 | −0.26 | 0.34 |
| 2. Emotional state | 0.30 | 0.61 | 0.03 |
| (global) | | | |
| 14. Feel sad | 0.20 | 0.71 | 0.08 |
| 15. Feel depressed | 0.20 | 0.81 | −0.04 |
| 16. Feel irritable | 0.06 | 0.87 | 0.04 |
| 17. Feel in a bad mood | 0.04 | 0.87 | 0.08 |
| 18. Feel angry | 0.11 | 0.85 | −0.04 |
| 19. Feel annoyed | 0.11 | 0.77 | 0.11 |
| 20. Feel anxious | 0.14 | 0.53 | 0.22 |
| 21. Feel stressed | −0.12 | 0.89 | 0.14 |
| 6. Sexual activities | 0.18 | 0.04 | 0.81 |
| (global) | | | |
| 28. Sexual intercourse | 0.06 | 0.07 | 0.87 |
| (enjoyment) | | | |
| 29. Sexual intercourse | −0.15 | 0.26 | 0.78 |
| (guilt at avoiding) | | | |

Descriptive Statistics are showing in the following Tables for scoring of pain endpoints: 28-day Average vs 7 ‘Worst-Days’.

TABLE 12
28-Day Average

| | Statistics of the Scaled Response | | | |
|---|---|---|---|---|
| Item | n | Mean (SD) | Q1-Q3 | Min, Max |
| 1. Worst pain (overall) | 269 | 5.17 (2.53) | 3.25-7.14 | 0.17-10 |
| 2. Worst constant pain | 269 | 4.22 (2.66) | 2.00-6.29 | 0-10 |
| 3. Worst short-term pain | 269 | 5.11 (2.63) | 3.00-7.14 | 0.17-10 |

TABLE 13
Worst 7 Days

| | Statistics of the Scaled Response | | | |
|---|---|---|---|---|
| Item | n | Mean (SD) | Q1-Q3 | Min, Max |
| 1. Worst pain (overall) | 269 | 5.17 (2.53) | 3.25-7.14 | 0.17-10 |
| 2. Worst constant pain | 269 | 4.22 (2.66) | 2.00-6.29 | 0-10 |
| 3. Worst short-term pain | 269 | 5.11 (2.63) | 3.00-7.14 | 0.17-10 |

Correlations between 28-day average scores for ESD items and PGI-S, EIS Item 1 (Physical activity) and EIS Item 2 (Emotional well-being) were compared to average 7 worst days scores. The strength of association with global impressions of patient severity and patient-reported impact are similar for items 1, 2 & 3 (indicating that no one item is more predictive of severity/impact). The correlations also indicate that there is no difference between 28-day average score and average 7 worst days score.

TABLE 14
Scoring of Pain Endpoints: 28-day Average vs 7
‘Worst-Days’ - Relationship to Impact

| | | | EIS Item 1: | EIS Item 2: |
|---|---|---|---|---|
| Measure- | | | Physical | Emotional |
| ment | Score | PGI-S | Impact | Impact |
| ESD Item 1: | 28-day Average | 0.68 | 0.62 | 0.63 |
| Worst Pain | Worst consec- | 0.72 | 0.63 | 0.62 |
| | utive 7 days | | | |
| ESD Item 2: | 28-day Average | 0.66 | 0.60 | 0.59 |
| Constant Pain | Worst consec- | 0.69 | 0.60 | 0.58 |
| | utive 7 days | | | |
| ESD Item 3: | 28-day Average | 0.66 | 0.61 | 0.61 |
| Short-term Pain | Worst consec- | 0.69 | 0.60 | 0.58 |
| | utive 7 days | | | |

To investigate the distinction between average 7 worst days score and dysmenorrhea, the number of bleeding days (≧light bleeding) in the “7-day worst” score were calculated. In 32% of patients, the 7 worst days of pain did not overlap at all with their menstrual period. In an additional 20% of patients, the overlap was only 1 or 2 days.

TABLE 15
Scoring of Pain Endpoints: 7 ‘Worst-Days’ -
Overlap with Dysmenorrhea

| Number of days | Number (%) of patients (N = 205) | | |
|---|---|---|---|
| where bleeding | ESD Item 1: | ESD Item 2: | ESD Item 3: |
| occurred | Worst Pain | Constant Pain | Short-term Pain |
| 0 | 65 (31.71%) | 62 (30.24%) | 67 (32.68%) |
| 1 | 19 (9.27%) | 16 (7.80%) | 21 (10.24%) |
| 2 | 22 (10.73%) | 22 (10.73%) | 23 (11.22%) |
| 3 | 20 (9.76%) | 25 (12.20%) | 19 (9.27%) |
| 4 | 26 (12.68%) | 26 (12.68%) | 24 (11.71%) |
| 5 | 21 (10.24%) | 19 (9.27%) | 16 (7.80%) |
| 6 | 16 (7.80%) | 16 (7.80%) | 17 (8.29%) |
| 7 | 16 (7.80%) | 19 (9.27%) | 18 (8.78%) |

1. A patient reported outcome questionnaire (PRO) for patients who have or are suspected of having endometriosis selected from the PRO shown in FIG. 1 and the PRO shown in FIG. 2.

2. The PRO of claim 1, wherein the PRO measures endometriosis-related pain and comprises a diagram the clearly highlights the areas in which patients with endometriosis typically experience pain.

3. The PRO of claim 1, wherein the PRO measures both chronic and acute endometriosis-related pain on a multi-point scale.

4. A method of diagnosing a patient suffering from endometriosis comprising: providing the PRO of claim 1 to a patient who has or is suspected of having endometriosis.

5. A method of treating a patient suffering from endometriosis comprising: providing the PRO of claim 1 to a patient who has or is suspected of having endometriosis, and administering a treatment for endometriosis.

6. A method of monitoring the treatment of a patient suffering from endometriosis comprising the steps of: providing a patient with a first PRO adapted for patients who have or are suspected of having endometriosis, administering a treatment for endometriosis, providing the patient with a second PRO who have or are suspected of having endometriosis.

7. The PRO of claim 1, wherein the PRO is designed to measure endometriosis-related pain and comprises a diagram the clearly highlights the areas in which patients with endometriosis typically experience pain.

8. The PRO of claim 1, wherein the PRO is designed to measure both chronic and acute endometriosis-related pain on a multi-point scale.

9. The PRO of claim 1, wherein the PRO is an Endometriosis Symptom Diary (ESD), designed for use by a patient in reporting report endometriosis symptoms.

10. The PRO of claim 9, wherein the ESD is in an electronic format.

11. The PRO of claim 9 wherein ESD is divided into 5 domains of investigation for assessing endometriosis-related pain.

12. The PRO of claim 11 wherein domains of investigation are Endometriosis pain, Constant pain, Short-term pain, Pain associated with menstruation, and Dyspareunia.

13. The PRO of claim 1 wherein patient is reporting impact of endometriosis symptoms onto life occurred the past seven days into an Endometriosis Impact Scale (EIS).

14. The PRO of claim 13, wherein the EIS is in an electronic format.

15. The PRO of claim 13 wherein the Endometriosis Impact Scale (EIS) consists into 6 domains of investigation impact of endometriosis-related pain onto patient life.

16. The PRO of claim 15 wherein the domains of investigation are Physical activities, Emotional well-being, Social and leisure activities, Work and study, Household activities and Sexual activities.

17. A method of treating a patient suffering from endometriosis comprising the steps of:
providing a patient who has or is suspected of having endometriosis with the PRO of claim 1 in the form of an Endometriosis Symptom Diary (ESD),
analyzing input provided by the patient in the ESD, and
administering treatment for endometriosis to the patient based on the results of that analysis.

18. The method of claim 17, wherein the input is analyzed using a diagram that clearly highlights the areas in which patients with endometriosis typically experience pain.

19. The method of claim 17, wherein the ESD is in an electronic format.

20. The method of claim 17, wherein the input provided in the ESD is analyzed using a computer.
